(19) United States
(12) Patent Application Publication
(10) Pub. No.: US 2010/0162964 A1
(43) Pub. Date: Jul. 1, 2010

(54) Process for polycross breeding Manila clam pedigree

(75) Inventors: Xiwu Yan, Dalian (CN); Yuehuan Zhang, Dalian (CN); Jun Liang, Dalian (CN); Zhongming Huo, Dalian (CN); Feng Yang, Dalian (CN); Guofan Zhang, Dalian (CN)

Correspondence Address: ZHEN ZHENG LU, 1730 HUNTINGTON DRIVE #304, DUARTE, CA, 91010, US

(73) Assignee: Dalian Fisheries University

(21) Appl. No.: 12/344,603

(22) Filed: Dec. 29, 2008

(51) Int. Cl. A01K 61/00; A01K 67/00; A01K 67/033

(52) U.S. Cl. 119/236; 119/234; 800/8

(57) ABSTRACT

A process for polycross breeding a Manila clam pedigree, includes: providing present Manila clam pedigrees of different shell color types as parents; ecologically maturing the parents; reciprocally hybridizing the parents by every two of different shell color types to obtain offsprings thereof; nurturing the offsprings until gonads thereof mature synchronously; and hybridizing the matured offsprings with each other to obtain at least a four-way cross pedigree accumulating all valuable genes of the parents.

### BACKGROUND OF THE PRESENT INVENTION

1. Field of Invention

The present invention relates to a breeding process, and more particularly to a process for polycross breeding a Manila clam pedigree marked by shell colors.

2. Description of Related Arts

Manila clam has various shell shapes, colors and patterns, and therefore was classified into different species by many taxonomists. The shell colors of marine bivalve were ignored as mere secretion product in past. Actually, the shell colors of bivalve does not only relate to ecology and behavior, but also relate to phenotypic character of growth and survival. At present, researchers study direct selective breeding of Manila clam shell color strains and two-way cross breeding thereof (Yan Xiwu, 2002-2008; Zhang Yuehuan, 2005-2008). The researchers combine the qualitative character, the quantity character and the economic character thereof effectively, and breed Manila clam shell color strains of high production and high stress resistance. Meanwhile, the researchers study the genetic mechanism of shell color of Manila clam (Taki, 1941; Richardson, 1977-1978; Gerard, 1978; Peignon, 1995; Yan Xiwu, 2002-2008; Zhang Yuehuan, 2005-2008). However, although there are some research reports about the polymorphism of shell color of Manila clam, there are no reports about polycross breeding of Manila clam pedigrees of different shell colors.

Selective breeding is an efficient method of breeding new varieties, and cross breeding is an important method of breeding species. Polycross breeding combines valuable genes of several parent varieties by a series of cross breeding. Additionally, Polycross breeding is applied widely to terrestrial animals and plants, but there are few research reports about marine animals. Chinese patents 2008100134243.6 and 200810013427.X disclose methods of indica hybrid and convergent cross breeding of Manila clam from different geographical populations. Chinese patents CN101288387A and CN101292635A disclose breeding methods of selective pedigrees and cross pedigrees of Manila clam, wherein the selective pedigrees and cross pedigrees are obtained by directive breeding and two-way cross breeding materials of Manila clam pedigrees of different shell colors.

### SUMMARY OF THE PRESENT INVENTION

An object of the present invention is to provide a process for breeding a Manila clam pedigree, which applies polycross breeding to present Manila clam pedigrees of different shell colors to obtain several new hybridized species.

Another object of the present invention is to provide a process for breeding a Manila clam pedigree having improved phenotyptic characters and high heterozygosity, in such a manner that the process improves variety of the present Manila clam, and increases germplasm thereof.

Another object of the present invention is to provide a process for breeding a Manila clam pedigree having beautiful appearance and high economic character, in such a manner that the process has great value in culture industry.

Another object of the present invention is to provide a process for breeding a Manila clam pedigree, wherein the process adopts selective pedigrees and cross pedigrees as parents to obtain a polycross pedigree by polycross breeding method.

Another object of the present invention is to provide a Manila clam pedigree bred according to the above process, wherein the Manila clam pedigree has improved phenotyptic character and high heterozygosity, and is valuable in culture industry.

Accordingly, in order to accomplish the above objects, the present invention provides a process for polycross breeding a Manila clam pedigree, comprising:

providing present Manila clam pedigrees of different shell color types as parents;

ecologically maturing the parents;

reciprocally hybridizing the parents by every two of different shell color types to obtain offsprings thereof;

nurturing the offsprings until gonads thereof mature synchronously; and

hybridizing the matured offsprings with each other to obtain at least a four-way cross pedigree accumulating all valuable genes of the parents.

Therefore the hybridized pedigree has all valuable genes of the present Manila clam pedigrees, and accordingly is valuable in culture industry.

The present invention also provides a polycross Manila clam pedigree bred according to the above process.

These and other objectives, features, and advantages of the present invention will become apparent from the following detailed description, and the appended claims.

### DETAILED DESCRIPTION OF THE PREFERRED EMBODIMENT

A process for polycross breeding a Manila clam pedigree according to a preferred embodiment of the present invention is illustrated, comprising:

providing present Manila clam pedigrees of different shell color types as parents;

reciprocally hybridizing the parents by every two of different color types to obtain offsprings thereof;

nurturing the offsprings until gonads thereof mature; and

hybridizing the matured offsprings with each other to obtain at least a four-way cross pedigree accumulating all valuable genes of the parents.

The process according to the present invention provides an operable and efficient way to achieve a polycross pedigree having improved characters of fast growth and high stress resistance, in such a manner that the polycross pedigree has greater economic value in culture industry.

According to the present invention, the process further comprises hybridizing the matured offsprings with the present Manila clam pedigrees of different shell colors to obtain at least a three-way cross pedigree accumulating all valuable genes of the present Manila clam pedigrees.

According to the present invention, the process further comprises ecologically maturing the parents before reciprocally hybridizing, so as to make gonads of the parents mature synchronously.

The process according to the present invention adopts ecologically maturing the parents to conquer a reproductive and physiological difference between pedigrees of different shell colors, promote the gonads to develop synchronously, and enhance eggs and sperms to emit.

Particularly, according to a preferred embodiment of the present invention, ecologically maturing comprises: disposing the parents into an ecological shrimp pond, and bottom sowing culturing the parents, until the gonads of the parents of different shell colors mature synchronously.

Particularly, according to another preferred embodiment of the present invention, ecologically maturing comprises: disposing the parents into a natural sea area, and bottom sowing culturing the parents, until the gonads of the parents of different shell colors mature synchronously.

According to the present invention, the process further comprises: after maturing and before reciprocally hybridizing, identifying sexes of the parents, and stimulating the parents by shade drying and water flushing to make the parents execute spawning and spermiate synchronously.

Identifying a sex of parents comprises: locating a gonad position of the parents; drilling on a shell thereof to reach to the gonad position; picking out sperms or eggs with a disinfected microsyringe; adding the sperms or eggs into seawater; and observing under microscope. The eggs diffuse in the seawater, and look round or pear under microscope; while the sperms look like smog in the seawater, and look like tadpole under microscope. Preferably, the present invention adopts an electric drill whose diameter is 0.6 mm.

Particularly, according to a preferred embodiment of the present invention, nurturing the offsprings comprises: disposing the offsprings into an ecological shrimp pond, and bottom sowing culturing the offsprings, until the gonads of the offsprings of different shell colors mature synchronously.

Particularly, according to another preferred embodiment of the present invention, nurturing the offsprings comprises: disposing the offsprings into a natural sea area, and bottom sowing culturing the offsprings, until the gonads of the offsprings of different shell colors mature synchronously.

Comparing with the present Manila clam varieties, a polycross individual has much higher heterozygosity, and characters of fast growth, high stress resistance and beautiful appearance.

The process according to the present invention adopts population hybridization, so as to avoid a drawback that hybridization in individual has relative single genotype, enrich genotype of the hybridzied individuals, and obtain plenty of offsprings more easily. Additionally, the hybridized individuals have faster growth and better survival than the parents, therefore represent an ultra-parent effect. Since the polycross individuals accumulate improved genes of different shell color strains, growth speed thereof increases 10˜20%, and survival rate thereof increases 60˜180%. The process according to the present invention provides a steady base for developing new polycross varieties of Manila clam.

Generally, the process according to the present invention comprises: providing Manila clam pedigrees of different shell colors as parents; ecologically maturing the parents of different shell color pedigrees; reciprocally hybridizing the parents by every two shell color pedigrees to obtain offsprings thereof; nurturing the offsprings; hybridizing the offsprings by polycross to obtain polycross offsprings accumulating all valuable genes of the parents of different shell color strains.

The present invention also provides a polycross Manila clam pedigree bred according to the above process.

### EXAMPLE 1

Step 1: in 2005, providing healthy and undamaged individuals of second instar as parents, wherein the parents come from south China Putian, and are classified into a background color strains (agate black clam, B; ocean red clam, R; and pearl white clam, W), a radial bands strains (two band red clam, Tr; and two band white clam, Tw), and a shell pattern strains (zebra clam, Z).

Step 2: ecologically maturing the parents by disposing the parents in an ecological shrimp pond of Zhuanghe sea village, and bottom sowing culturing the parents for 6˜12 months. Gonads of the parents mature synchronously in Autumn, 2006.

Step 3: identifying sexes of parents with an electric drill whose diameter is 0.6 mm. After identifying, by stimulating the parents by shade drying for 8 hours and water flushing for 1 hour, the parents execute spawning and spermiate synchronously. Then reciprocally hybridizing the parents by hybridizing groups of B×Z, R×Z, W×Z, Tr×Z, Tw×Z, B×W, and R×W in population. Each hybridizing group uses 200 parents to obtain two-way cross offsprings thereof.

Step 4: nurturing the two-way cross offspring in the ecological shrimp pond of Zhuanghe sea village, nurturing period ≧1 year. In Autumn, 2007, gonads of the two-way cross offspring mature synchronously.

Step 5: as the step 3, hybridizing the parents by hybridizing groups of F_{B}_{^{Z}}×F_{W}, F_{WZ}×F_{B}, F_{RZ}×F_{W}, F_{WZ}×F_{R} to obtain three-way cross offsprings F_{BZW} and F_{RZW}, and hybridizing the parents by hybridizing groups of F_{BZ}×F_{RW}, F_{R}×F_{BW}, to obtain a four-way cross offspring F_{BZRW}.

Growth speed of the hybridized Manila clams increases 10˜20%, and survival rate thereof increases 90˜180%.

### EXAMPLE 2

Step 1: in 2005, providing healthy and undamaged individuals of second instar as parents, wherein the parents come from south China Putian, and are classified into a background color strains (agate black clam, B; ocean red clam, R; and pearl white clam, W), a radial bands strains (two band red clam, Tr; and two band white clam, Tw), and a shell pattern strains (zebra clam, Z).

Step 2: ecologically maturing the parents by disposing the parents in a natural sea area of Zhuanghe sea village, and bottom sowing culturing the parents for 6˜12 months. Gonads of the parents mature synchronously in Autumn, 2006.

Step 3: identifying sexes of parents with an electric drill whose diameter is 0.6 mm. After identifying, by stimulating the parents by shade drying for 8 hours and water flushing for 1 hour, the parents execute spawning and spermiate synchronously. Then reciprocally hybridizing the parents by hybridizing groups of B×Z, R×Z, W×Z, Tr×Z, Tw×Z, B×W, and R×W in population. Each hybridizing group uses 200 parents to obtain two-way cross offsprings thereof.

Step 4: nurturing the two-way cross offspring in the natural sea area of Zhuanghe sea village, nurturing period ≧1 year. In Autumn, 2007, gonads of the two-way cross offspring mature synchronously.

Step 5: as the step 3, hybridizing the parents by hybridizing groups of F_{BZ}×F_{W}, F_{WZ}×F_{B}, F_{RZ}×F_{W}, F_{WZ}×F_{R} to obtain three-way cross offsprings F_{BZW} and F_{RZW}, and hybridizing the parents by hybridizing groups of F_{BZ}×F_{RW}, F_{RZ}×F_{BW}, to obtain a four-way cross offspring F_{BZRW}.

Growth speed of the hybridized Manila clams increases 10˜15%, and survival rate thereof increases 60˜120%.

One skilled in the art will understand that the embodiment of the present invention as described above is exemplary only and not intended to be limiting.

It will thus be seen that the objects of the present invention have been fully and effectively accomplished. It embodiments have been shown and described for the purposes of illustrating the functional and structural principles of the present invention and is subject to change without departure from such principles. Therefore, this invention includes all modifications encompassed within the spirit and scope of the following claims.

What is claimed is:

1. A process for polycross breeding a Manila clam pedigree, comprising:
providing present Manila clam pedigrees of different shell color types as parents;
reciprocally hybridizing the parents by every two of different color types to obtain offsprings thereof;
nurturing the offsprings until gonads thereof mature; and
hybridizing the matured offsprings with each other to obtain at least a four-way cross pedigree accumulating all valuable genes of the parents.

2. The process, as recited in claim 1, further comprising hybridizing the offsprings with the parents to obtain at least a three-way cross pedigree accumulating all valuable genes of the parents.

3. The process, as recited in claim 1, further comprising ecologically maturing the parents before reciprocally hybridizing, so as to make gonads of the parents mature synchronously.

4. The process, as recited in claim 2, further comprising ecologically maturing the parents before reciprocally hybridizing, so as to make gonads of the parents mature synchronously.

5. The process, as recited in claim 3, wherein ecologically maturing comprises: disposing the parents into an ecological shrimp pond, and bottom sowing culturing the parents, until the gonads of the parents of different shell colors mature synchronously.

6. The process, as recited in claim 4, wherein ecologically maturing comprises: disposing the parents into an ecological shrimp pond, and bottom sowing culturing the parents, until the gonads of the parents of different shell colors mature synchronously.

7. The process, as recited in claim 3, wherein ecologically maturing comprises: disposing the parents into a natural sea area, and bottom sowing culturing the parents, until the gonads of the parents of different shell colors mature synchronously.

8. The process, as recited in claim 4, wherein ecologically maturing comprises: disposing the parents into a natural sea area, and bottom sowing culturing the parents, until the gonads of the parents of different shell colors mature synchronously.

9. The process, as recited in claim 1, further comprising: after maturing and before reciprocally hybridizing, identifying sexes of the parents, and stimulating the parents by shade drying and water flushing to make the parents execute spawning and spermiate synchronously.

10. The process, as recited in claim 2, further comprising: after maturing and before reciprocally hybridizing, identifying sexes of the parents, and stimulating the parents by shade drying and water flushing to make the parents execute spawning and spermiate synchronously.

11. The process, as recited in claim 3, further comprising: after maturing and before reciprocally hybridizing, identifying sexes of the parents, and stimulating the parents by shade drying and water flushing to make the parents execute spawning and spermiate synchronously.

12. The process, as recited in claim 4, further comprising: after maturing and before reciprocally hybridizing, identifying sexes of the parents, and stimulating the parents by shade drying and water flushing to make the parents execute spawning and spermiate synchronously.

13. The process, as recited in claim 1, wherein nurturing the offsprings comprises: disposing the offsprings into an ecological shrimp pond, and bottom sowing culturing the offsprings, until the gonads of the offsprings of different shell colors mature synchronously.

14. The process, as recited in claim 2, wherein nurturing the offsprings comprises: disposing the offsprings into an ecological shrimp pond, and bottom sowing culturing the offsprings, until the gonads of the offsprings of different shell colors mature synchronously.

15. The process, as recited in claim 1, wherein nurturing the offsprings comprises: disposing the offsprings into a natural sea area, and bottom sowing culturing the offsprings, until the gonads of the offsprings of different shell colors mature synchronously.

16. The process, as recited in claim 2, wherein nurturing the offsprings comprises: disposing the offsprings into a natural sea area, and bottom sowing culturing the offsprings, until the gonads of the offsprings of different shell colors mature synchronously.

17. A polycross Manila clam pedigree bred by a process comprising:
providing present Manila clam pedigrees of different shell color types as parents;
reciprocally hybridizing the parents by every two of different color types to obtain offsprings thereof;
nurturing the offsprings until gonads thereof mature; and
hybridizing the matured offsprings with each other to obtain at least a four-way cross pedigree accumulating all valuable genes of the parents.

18. The polycross Manila clam pedigree, as recited in claim 17, wherein the process further comprises hybridizing the offsprings with the parents to obtain at least a three-way cross pedigree accumulating all valuable genes of the parents.

19. The polycross Manila clam pedigree, as recited in claim 17, wherein the process further comprises ecologically maturing the parents before reciprocally hybridizing, so as to make gonads of the parents mature synchronously.

20. The polycross Manila clam pedigree, as recited in claim 17, wherein the process further comprises: after maturing and before reciprocally hybridizing, identifying sexes of the parents, and stimulating the parents by shade drying and water flushing to make the parents execute spawning and spermiate synchronously.
